(19) United States
(12) Patent Application Publication
(10) Pub. No.: US 2024/0350570 A1
(43) Pub. Date: Oct. 24, 2024

(54) TOPICAL SKIN CARE PREPARATION COMPRISING EPHEDRA VIRIDIS

(71) Applicant: Daniel Tyler WHITING, Orem, UT (US)

(72) Inventor: Daniel Tyler WHITING, Orem, UT (US)

(21) Appl. No.: 18/640,405

(22) Filed: Apr. 19, 2024

Related U.S. Application Data
(60) Provisional application No. 63/460,536, filed on Apr. 19, 2023.

(51) Int. Cl. A61K 36/17; A61K 9/00; A61K 9/06; A61K 36/534; A61K 36/70; A61K 36/88

(52) U.S. Cl. CPC A61K 36/17; A61K 9/0014; A61K 9/06; A61K 36/534; A61K 36/70; A61K 36/88

(57) ABSTRACT

A method and related topical skin preparation comprising an extract from Ephedra viridis. The disclosed method and preparation treat fever blisters caused by herpes simplex virus and other skin-related inflammatory conditions.

### BACKGROUND

The present invention relates to a topical skin preparation for treating fever blisters and other skin-related inflammatory conditions, and more particularly a method and composition for treating herpes virus, herpes virus, mouth ulcers and other microbial infections.

Herpes simplex virus (HSV) commonly referred to as “herpes virus” or “herpes,” is a linear dsDNA virus that is a member of the Alphaherpesviridae subfamily. There are two common types of herpes: herpes simplex virus 1 (HSV-1) and herpes simplex virus 2 (HSV-2). HSV-1 is the most common culprit of orolabial herpes (a small percent of cases are attributed to HSV-2). It is important to note that orolabial HSV-1 infection is most commonly asymptomatic. When there are symptoms, the most common manifestation is the “cold sore” or fever blister.

These small fluid-filled blisters are often grouped together in patches and typically erupt along the border of the lips. Sometimes they appear around the nose or cheeks or inside the mouth, though they can appear anywhere on the skin or mucosa. After the blisters break, a scab can form that lasts for several days. Cold sores can heal over two to three weeks without leaving a scar; however, many individuals experience itching, burning, or tingling around the lips before a small, hard, painful spot appears and the blisters erupt. In some immune compromised individuals, the outbreak can last for months. In children, symptomatic orolabial HSV-1 infections often present as gingivostomatitis that leads to pain, halitosis, and dysphagia. In adults, it can present as pharyngitis and a mononucleosis-like syndrome.

Antiviral medications have been developed for oral administration in an attempt to slow down the activity of the virus within the blister. However, these antiviral medications require excessive costs for manufacturing of the pharmaceutical ingredients. The extensive use of viral drugs can lead to the development of some drug resistance, which in turn leads to treatment failure. Additionally, over-the-counter topical treatments for cold sores have been developed and include topical anesthetics, skin protectants, antiseptics, or herbal remedies. Most topical treatments attempt to reduce the pain and appearance of the cold sore; however, they usually have little effect on the duration of the cold sore and also require excessive manufacturing costs. As such, there is a need in the art to provide a cost-effective therapeutic compound to reduce the duration and scope of blisters resulting from the HSV.

### SUMMARY

The disclosed topical skin care preparation, or virus remedy preparation, is directed to particular methods and preparations of a topical skin composition that can be used to alleviate the symptoms that stem from viral infections with a plant-based, non-pharmaceutical product comprising Ephedra viridis. In particular, the inventor of the disclosed topical skin care preparation discovered that the Ephedra viridis indigenous to the Western United States, where it is a member of varied scrub, woodland, desert, and open habitats, possessed improved healing and skin care properties compared to other types of Ephedra plants, (e.g., Ma huang), grown elsewhere in the world. By way of example, the disclosed topical skin care preparation may be used to treat certain skin conditions ranging from skin-related inflammatory conditions to symptoms from an outbreak of HSV blisters.

In an embodiment, the disclosed preparation provides a method for treating a condition of the skin. The method comprising topically applying to skin a composition comprising an extract from Ephedra viridis. In particular, the method includes applying a therapeutically effective amount of a composition comprising an extract from Ephedra viridis. The method uses an effective amount of the composition comprising an extract from Ephedra viridis to treat fever blisters caused by HSV or other skin-related inflammatory conditions. The composition preferably has a dermatologically acceptable carrier for the plant, plant part, or extract of Ephedra viridis. The composition may further includes an oil, a wax, a moisturizing agent, a silicone containing compounds, a UV agent, a surfactant, and/or other ingredients identified in this specification or those known in the art for skin treatment. In particular aspects, the composition is formed as a cream, ointment, lotion, lip balm, spray, solution, gel, soap, serum, emulsion, anhydrous base, paste, etc.

In an embodiment, the extract from Ephedra viridis comprises a powdered substance formed from mechanically separated or isolated bark of the Ephedra viridis plant. The composition may be in powdered form (e.g., dried, lyophilized, particulate, etc.). Each Ephedra viridis plant extract may be individually obtained, ground, and dried to produce the powder substance. Preferably, the bark of the Ephedra viridis plant is mechanically separated from the stem and ground into powder form. The powder may then added be to the topical skin preparation.

In an embodiment, the composition further comprises an oil-wax base. The oil-wax base comprises at least one oil ingredient and at least one wax ingredient. The oil ingredient is a carrier oil and can be selected from the following ingredients: olive oil, coconut oil, shea butter, cocoa butter, grapeseed oil, canola oil, sweet almond oil, Jojoba oil, Argan oil, Tamanu oil, Rosehip oil, avocado oil, castor oil, petrolatum oil, and walnut oil. The wax ingredient can be selected from the following ingredients: beeswax, candelilla wax, carnauba wax, camphor, rice wax, cetyl alcohol, sunflower wax, lanolin wax, paraffin wax, berry wax, Myrica fruit wax, and Laurel wax. Some embodiments may comprise an oil base, without wax, or a wax base, without oil.

In an embodiment, the composition may further comprise an extract from Rumex crispus. The extract from Rumex crispus can be from a stout root of the Rumex crispus that is bright yellow below the bark. The stout root may be ground into a fine powder and mixed with other ingredients of the composition. The extract from Rumex crispus can be prepared from the leaves of the plant or prepared as a tincture to be combined with the extract of Ephedra viridis. The extract of Rumex crispus is used in combination with the Ephedra viridis extract and promotes an effective treatment of skin issues including psoriasis, eczema, and acne.

In an embodiment, the composition further comprises an essential oil. The essential oil can be used as an aroma component and may be selected from the following: camomile oil, cinnamon leaf oil, clove oil, Galbanum oil, juniper berry oil, labolanum oil, lavandin oil, linden blossom oil, Melissa oil, mint oil, olibanum oil, sage oil, and vetiver oil. It is also possible to use allyl amyl glycolate, ambroxan, benzyl acetate, benzyl acetone, bergamot oil, boisambrene forte, citronellol, clary sage oil, cyclamenaldehyde, cyclohexyl salicylate, cyclovertal, dihydromyrcenol, evernyl, fixolide NP, floramat geraniol, geranium oil bourbon, geranyl acetate, hedione, indole, iraldein gamma, irotyl, iso-E-super, lavandin oil, lemon oil, lilial, linalool, lyral, mandarin oil, orange oil, phenylacetic acid, phenylethyl alcohol, romillat, rose oxide, sandelice, vertofix coeur, α-hexylcinnamaldehyde, and β-damascone alone or in combination with another essential oil.

In an embodiment, the composition further comprises an extract from Plantago lanceolata, Plantago major, or plantain leaf. Plantain comprises astringent, antibacterial, antioxidant, and anti-inflammatory properties that help to soothe and heal skin. Plantain also promotes anti-ulcerogenic activity, accelerates wound healing, and supports digestive health.

Regarding the method of treating skin, the method comprising topically applying to skin a composition comprising an extract from Ephedra viridis, there is a first observation that there is a rapid reduction in pain. The pain remediation is continuous for the duration of time that the composition is applied to the infected area. Following the application of the composition, a slight swelling is observed followed by an increase in blood flow to the infected area. The duration and intensity of the slight swelling may vary for each individual; however, the slight swelling typically lasts between 4 to 12 hours.

Following the slight swelling, the infected area may no longer show signs of the infection for some individuals. For others, a healing processed is observed which ranges from 12 to 48 hours, depending on the individual, the size of the infected area, and the depth of infection. The damaged tissue is pushed to the surface of the skin as the skin layers are healed from the innermost layers to the outermost layers in a typical wound healing manner, i.e., following hemostasis and inflammation at the infected site, proliferation and remodelling are observed.

For other individuals, following the slight swelling, a clear liquid comprising white blood cells, presumably comprising the dead and dying virus, is transferred through the tissue to the surface of the skin. As the virus appears to be expelled from the infected area, there is no observation of a wound opening (or subsequent split or crack), and the following the clear bodily fluid or exudate is transmitted through the skin without damaging or disrupting the epidermis.

In an embodiment, a topical skin preparation comprising an extract from Ephedra viridis is provided. The preparation can have a dermatologically acceptable vehicle or carrier for the plant, plant part, or extract thereof. The preparation of the present invention can also take the form of topically spreadable preparations, sprayable preparations, aerosolized preparations, injectable preparations, edible preparations, or preparations in tablet, gel cap, or pill form. Kits that include the compositions of the present invention are also contemplated. In certain embodiments, the composition is comprised in a container. The container can be a bottle, dispenser, or package. In certain aspects, the composition is dispensed in a spray, dollop, or liquid form. The container can include indicia on its surface for identifying words, abbreviations, pictures, and/or symbols associated with the disclosed preparation.

In an embodiment, the extract from Ephedra viridis comprises a powdered substance formed from mechanically separated or isolated bark of the Ephedra viridis plant. The topical skin preparation may comprise a carrier oil base and/or a wax base as described above. The topical skin preparation may further comprise an extract from Rumex crispus and/or a plantain extract. The topical skin preparation may also comprise an essential oil.

The benefits of the topical skin preparation and associated compositions and methods include a significant amount of pain reduction, an effective and biocompatible skin treatment, and an affordable solution without manufactured pharmaceutical ingredients.

These and other features, aspects, and advantages of the present disclosure will become better understood regarding the following description, appended claims, and accompanying drawing(s).

### BRIEF DESCRIPTION OF THE DRAWING(S)

The FIGURE(S) illustrate exemplary configurations and/or methods of the disclosed viral remedy preparation, and in no way limit the structures or configurations according to the present disclosure.

FIG. 1 is a flow chart showing a preferred process of the disclosed virus remedy.

### DEFINITIONS

“Ephedra viridis” is a species of Ephedra and is known by the common names of Mormon tea, Brigham tea, Indian tea, and green Ephedra. It is indigenous to the Western United States. Although the specification consistently refers to Ephedra viridis, other species of Ephedra, such as Ephedra nevadensis, may be used in place of, or in combination with, Ephedra viridis. It is worth mentioning that the stems of Ephedra viridis are generally more erect and dark green on healthy plants, while those of Ephedra nevadensis are generally wider spreading at divergent angles and more of a gray-green color. However, both species are effective in treating viral infections. Additionally, both species of Ephedra viridis and Ephedra nevadensis are referred to as Mormon Tea.

The term “carrier oil” means an oil that is used as a base ingredient that is safe enough to use directly on the skin and can be used to dilute essential oils. Preferably, carrier oils make up a majority ingredient of the final topically applied preparation.

The term “wax” includes a diverse class of organic compounds that are lipophilic, malleable solids near ambient temperatures. They include higher alkanes and lipids, typically with melting points above about 40° C., melting to give low viscosity liquids.

“Rumex crispus” is a species of Polygonaceae and is known by the common names of Curly Dock, Oseille Crdpue, Acedera, Yellowdock, Rumex obstusifolius, Narrow Dock, Rumex, Broad-Leaved Dock, Chukkah, Parelle Sauvage, Field Sorrel, Rumex crispus, Amalvelas, Patience Crépue, Herbe à Cochons, Romaza, Sheep Sorrel, Lengua de Vaca, Sour Dock, Curled Dock.

Essential oils include oils derived from trees, herbs, flowers, and other plants. Such oils are typically present as tiny droplets between the plant's cells, and can be extracted by several methods known to those of skill in the art (e.g., steam distilled, enfleurage, maceration, solvent extraction, or mechanical pressing). Essential oils are concentrated plant extracts that are insoluble in water and are soluble in alcohol, ether, fixed oils (vegetal), and other organic solvents.

The term “mechanically separate” generally refers to mechanically disrupting the tissue of a plant in order to separate intact cells of certain organs and isolate distinct parts of the plant. Mechanical separation may be performed manually or automatically (e.g., with a tool or machine) by acts of pealing, grinding, cutting, filtering, and the like.

The term “topical skin preparation” includes compositions and formulations that are suitable for topical application on human skin. Such preparations are typically dermatologically-acceptable and do not demonstrate undue toxicity, allergic response incompatibility, and the like, when applied to the skin. Topical skin care preparations of the disclosed methods and compositions can have a selected viscosity to avoid significant liquifying or dripping after application to skin.

The term “extract” is a substance made by extracting a part of a raw material, such as a plant, often by using a solvent such as ethanol, oil, or water. Extracts may be formed as tinctures, absolutes, or in powder form.

### DETAILED DESCRIPTION OF VARIOUS EMBODIMENTS

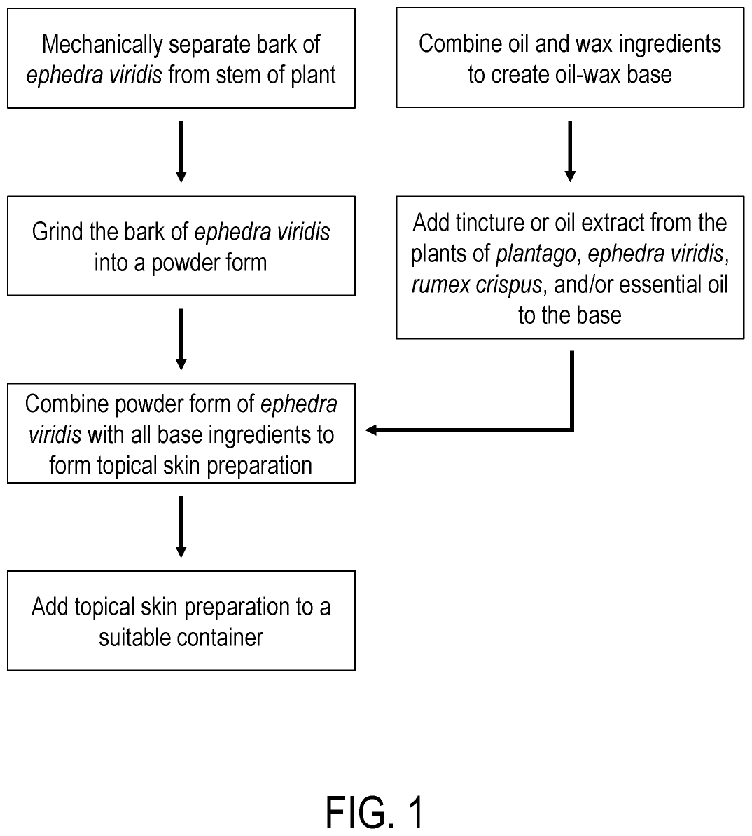

FIG. 1 is a flow chart showing an exemplary process of the disclosed virus remedy. The process includes mechanically separating the bark of the Ephedra viridis plant from the stem of the Ephedra viridis plant. Following the mechanical separation or isolation of the Ephedra viridis bark from the stem, the bark is ground into a powder form. In an embodiment, the powdered form of bark is dried, lyophilized, particulate, etc. Finely grinding the plant material creates more surface area for the Ephedra viridis extract to interact with the skin, leading to an efficient, uniform distribution of the extract throughout the virus remedy composition or topical skin preparation. Grinding is typically performed in a laboratory mill, utilizing a blade, grinding balls, and/or sieve to reduce the plant into fine particles. The Ephedra viridis plant and its powdered derivative forms are skin-friendly and have a low risk of causing allergies, sensitivity, or skin irritation. An effective amount of the disclosed composition comprising an extract from Ephedra viridis may be applied to treat fever blisters caused by HSV or other skin-related inflammatory conditions.

The composition of the disclosed virus remedy, or topical skin preparation, can include from about 0.01% to about 5% by weight of said extract from Ephedra viridis. Amounts and ranges within and outside the stated range are also contemplated, e.g., ranges and values selected from the following percentages: 0.0001%, 0.0005%, 0.001%, 0.005%, 0.05%, 0.1%, 0.5%, 1%, 2%, 5%, 6%, 7%, 8%, 9%, 10%, 15%, 20%, and 30%.

To facilitate ease of application to the skin, the virus remedy, which is observed as a topical skin preparation with respect to FIG. 1, may be combined with a base and other supplemental ingredients. The base ingredient may be formed as an oil-wax base. The oil-wax base comprises at least one carrier oil ingredient and at least one wax ingredient. The composition of the disclosed virus remedy, or topical skin preparation, can include from about 95% to about 99.99% by weight of said oil-wax base. Amounts and ranges within and outside the stated range are also contemplated, e.g., ranges and values selected from the following percentages: 70%, 80%, 85%, 90%, 91%, 92%, 93%, 94%, 95%, 98%, 99%, 99.5%, 99.9%, 99.95%, 99.995%, 99.999%, 99.9995%, and 99.9999%.

Carrier oils, also known as base oils, are fat-based oils that are typically cold-pressed from various plant sources, such as almonds or coconuts. They are called ‘carrier oils’ because they are used to dilute concentrated ingredients, such as essential oils, and carry them safely onto the skin when applied. Carrier oils are rich in vitamins, minerals, and fatty acids that are nourishing and are safe to apply directly to the skin. The nourishing properties of carrier oils facilitate in hydrating and protecting the skin. The carrier oil can be a synthetic or natural oil derived from seeds, nuts, fruits, flowers, plant-based materials, or any combinations thereof. The carrier oil ingredient may be selected from the following ingredients: white mineral oil, Tamanu oil, olive oil, Jojoba oil, calendula oil, canola oil, castor oil, almond oil, sweet almond oil, coconut oil, hydrogenated coconut oil, caprylic/capric triglyceride (derived from coconut oil and glycerine), grapeseed oil, corn oil, soybean oil, Argon oil, Rosehip oil, petrolatum oil, avocado oil, sunflower oil, pomegranate seed oil, hemp seed oil, shea butter, cocoa butter, and walnut oil.

Waxes form an important group of ingredients for the manufacture of personal care products and decorative cosmetics. Waxes are complex, chemical mixtures of hydrocarbons and fatty acids combined with esters. Waxes provide structural support, are less greasy than other structuring agents, and are resistant to moisture, oxidization, and microbial attacks. Examples of suitable wax ingredients include, but are not limited to, beeswax, candelilla wax, carnauba wax, camphor, rice wax, cetyl alcohol, sunflower wax, lanolin wax, paraffin wax, berry wax, Myrica fruit wax, and Laurel wax. In a preferred embodiment, the wax ingredient is a natura beeswax produced by bees. Beeswax is a thickening agent that thickens the oil portion of solid products (like lip balms), giving them structure and keeping them solid. The disclosed waxes also provide an emollient that smooths and softens the skin. As an example, candelilla wax is a wax derived from the candelilla plant which possesses lubricating properties that are useful in topical products.

The disclosed topical skin preparation may also include an extract of Plantago or Plantain. Due to its emollient qualities, plantain helps calm skin inflammation. Plantain also has a natural anti-inflammatory and bactericidal effect on the skin. Plantain is an astringent with soothing and healing properties that reduces oil secretions of the skin. Additionally, the topical skin preparation may also comprise an extract of Rumex crispus as an effective ingredient. Rumex crispus extract is an astringent and antibacterial ingredient that helps restore and purify skin.

The disclosed process in FIG. 1 also suggests the process of adding an essential oil to form the topical skin preparation. Each essential oil has a unique composition of chemicals, and this variation affects the smell, absorption, and response with the body. Non-limiting examples of essential oils that can be used include anise oil, balsam oil, bergamot oil, black pepper oil, cedar oil, chamomile oil, clary sage oil, clove oil, coriander oil, cypress oil, Eucalyptus oil, evening primrose oil, fennel oil, frankincense oil, geranium oil, ginger oil, grapefruit oil, jasmine oil, juniper oil, lavender oil, lemon oil, lemongrass oil, lime oil, macadamia nut oil, mandarin oil, marjoram oil, myrrh oil, neroli oil, orange oil, patchouli oil, pepper oil, petitgrain oil, pimento berries oil, pine oil, rose oil, rose otto oil, rosemary oil, rosewood oil, sage oil, sandalwood oil, sea fennel oil, sesame oil, Spanish rosemary oil, Spanish sage oil, spearmint oil, spikenard oil, tea tree oil, thyme oil, vetiver oil, wintergreen oil, or ylang ylang. Other essential oils known to those of skill in the art are also contemplated as being useful in the compositions described herein.

In particular aspects, the disclosed composition is formed as a cream, ointment, lotion, lip balm, spray, solution, gel, soap, serum, emulsion, anhydrous base, paste, etc.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

This example is used to demonstrate an exemplary composition of the disclosed virus remedy that may be used as a lip balm for applying the topical skin preparation to the skin on or about the lips. A solution of oil and wax is warmed and combined to create the base for the remedy to be topically applied to the infected area. A tincture or oil extract from the plants commonly referred to as Plantain, Yellow Dock, and Mint oil is added to the base. The plant of Ephedra viridis is individually obtained, and the bark from the Ephedra viridis is mechanically separated from the stem and ground into a dried powder form. The powder is then added to the base. The combined mixture of all components is then allowed to cool and added to a lip balm container for topical application. By way of example, the container has a capacity of 0.15 fl. oz. The topical skin preparation comprises approximately 0.14 fl. oz. of the base, 0.001 oz. of Ephedra viridis extract, and 0.00025 fl. oz. of the Mint oil. The Ephedra viridis and Mint oil are evenly distributed throughout the base within the container for a uniform allocation of the extract.

It is to be understood that not necessarily all objects or advantages may be achieved under any embodiment of the disclosure. Those skilled in the art will recognize that the disclosed virus remedy may be embodied or carried out in a manner that achieves or optimizes one advantage or group of advantages as taught herein without achieving other objects or advantages as taught or suggested herein.

The skilled artisan will recognize the interchangeability of various disclosed features. Besides the variations described herein, other known equivalents for each feature can be mixed and matched by one of ordinary skill in this art to create and use a topical skin preparation under principles of the present disclosure. It will be understood by the skilled artisan that the features described herein may be adapted to other methods and types of therapeutic and cosmetic applications.

It is intended that the present disclosure should not be limited by the disclosed embodiments described above and may be extended to other applications that may employ the features described herein.

1. A method of treating skin, the method comprising topically applying to skin a composition including an extract from Ephedra viridis.

2. The method of claim 1, wherein fever blisters caused by herpes simplex virus or other skin-related inflammatory conditions are treated with a therapeutically effective amount of the composition.

3. The method of claim 1, wherein the composition is a cream or a lotion.

4. The method of claim 1, wherein the composition comprises 0.0001% to 5%, by weight, of the extract from Ephedra viridis.

5. The method of claim 1, wherein the composition further comprises an oil-wax base, the oil-wax base including at least one oil ingredient and at least one wax ingredient.

6. The method of claim 5, wherein the at least one wax ingredient is selected from the group consisting of beeswax, candelilla wax, carnauba wax, camphor, rice wax, cetyl alcohol, sunflower wax, lanolin wax, paraffin wax, berry wax, Myrica fruit wax, and Laurel wax.

7. The method of claim 5, wherein the at least one oil ingredient is selected from the group consisting of olive oil, coconut oil, shea butter, cocoa butter, grapeseed oil, canola oil, sweet almond oil, Jojoba oil, Argan oil, Tamanu oil, Rosehip oil, avocado oil, castor oil, petrolatum oil, and walnut oil.

8. The method of claim 1, wherein the extract from Ephedra viridis comprises a powdered substance formed from isolated bark of the Ephedra viridis.

9. The method according to claim 1, wherein the composition further comprises an extract from Rumex crispus.

10. The method according to claim 1, wherein the composition further comprises an essential oil.

11. The method according to claim 1, wherein the composition further comprises a plantain leaf extract.

12. A topical skin preparation comprising an extract from Ephedra viridis.

13. The topical skin preparation according to claim 12, wherein the preparation further comprises an oil-wax base, the oil-wax base having at least one carrier oil ingredient and at least one wax ingredient.

14. The topical skin preparation according to claim 13, wherein the at least one wax ingredient is selected from the group consisting of beeswax, candelilla wax, carnauba wax, camphor, rice wax, cetyl alcohol, sunflower wax, lanolin wax, paraffin wax, berry wax, Myrica fruit wax, and Laurel wax.

15. The topical skin preparation according to claim 13, wherein the at least one carrier oil ingredient is selected from the group consisting of olive oil, coconut oil, shea butter, cocoa butter, grapeseed oil, canola oil, sweet almond oil, Jojoba oil, Argan oil, Tamanu oil, Rosehip oil, avocado oil, castor oil, petrolatum oil, and walnut oil.

16. The topical skin preparation according to claim 12, wherein the extract from the Ephedra viridis includes a powdered substance formed from isolated bark of the Ephedra viridis, the isolated bark being mechanically separated from one or more stems of the Ephedra viridis.

17. The topical skin preparation according to claim 12, wherein the preparation further comprises an extract from Rumex crispus.

18. The topical skin preparation according to claim 12, wherein the preparation further comprises an essential oil.

19. The topical skin preparation according to claim 12, wherein the preparation further comprises a plantain leaf extract.

20. A topical skin preparation comprising:
an extract from Ephedra viridis;
an oil-wax base having least one carrier oil ingredient and at least one wax ingredient;
wherein the at least one wax ingredient is selected from the group consisting of beeswax, candelilla wax, carnauba wax, camphor, rice wax, cetyl alcohol, sunflower wax, lanolin wax, paraffin wax, berry wax, Myrica fruit wax, and Laurel wax;
wherein the at least one carrier oil ingredient is selected from the group consisting of olive oil, coconut oil, shea butter, cocoa butter, grapeseed oil, canola oil, sweet almond oil, Jojoba oil, Argan oil, Tamanu oil, Rosehip oil, avocado oil, castor oil, petrolatum oil, and walnut oil;
wherein the extract from the Ephedra viridis includes a powdered substance derived from bark isolated from the Ephedra viridis.
